(19) United States
(12) Patent Application Publication
(10) Pub. No.: US 2013/0040990 A1
(43) Pub. Date: Feb. 14, 2013

(54) SYNTHESIS OF DGJNAc FROM D-GLUCURONOLACTONE AND USE TO INHIBIT ALPHA-N-ACETYLGALACTOSAMINIDASES

(71) Applicants: George William John Fleet, Oxford (GB); Terry Douglas Butters, Oxford (GB)

(72) Inventors: George William John Fleet, Oxford (GB); Terry Douglas Butters, Oxford (GB)

(73) Assignee: The Chancellor, Masters and Scholars of the University of Oxford

(21) Appl. No.: 13/576,453

(22) PCT Filed: Jan. 28, 2011
(86) PCT No.: PCT/IB2011/000380

§ 371 Date: Oct. 19, 2012

Related U.S. Application Data
(60) Provisional application No. 61/282,393, filed on Feb. 2, 2010.

(51) Int. Cl. C07D 211/56; A61K 31/445; A61P 3/00; C12N 9/99

(52) U.S. Cl. 514/315; 435/184; 546/242; 546/220

(57) ABSTRACT

A convenient and scalable synthesis of DGJNAc ID from D-glucuronolactone in an overall yield of 20% is provided. DGJNAc is the first highly potent and specific competitive inhibitor of GalNAcases. DGJNAc ID is also a competitive inhibitor of -hexosaminidases. Synthesis and activity of L-DGJNAc IL is also shown. The use of DGJNAc as a potent and specific inhibitor of GalNAcases will allow useful investigation and treatment of a number of diseases, including Schindler Disease.

### CROSS-REFERENCE TO RELATED APPLICATIONS

This application claims the benefit of U.S. Provisional Patent Application No. 61/282,393, filed Feb. 2, 2010, the entire disclosures of which are incorporated by reference.

### FIELD

The present disclosure relates in general to the use and synthesis of iminosugars for medical purposes and, in particular, to the use of iminosugars for inhibiting α-N-acetylgalactosaminidases (GalNAcases) or β-hexosaminidases, as well as treatments for diseases associated with these enzymes.

### SUMMARY

According to one embodiment, the current invention discloses a method for synthesizing DGJNAc or a DGJNAc derivative from D-glucuronolactone. Synthesis of DGJNAc comprises introducing nitrogen at C5 of glucuronolactone, inversion of configuration of the hydroxyl group at C3 and formation of the piperidine ring by introduction of nitrogen between C6 and C2.

According to another embodiment, numerous novel DGJNAc derivatives are disclosed. These compositions include a compound of the formula,

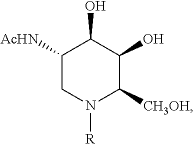

or a pharmaceutically acceptable salt thereof, wherein R is selected from the group consisting of substituted or unsubstituted alkyl groups, substituted or unsubstituted cycloalkyl groups, substituted or unsubstituted aryl groups, and substituted or unsubstituted oxaalkyl groups; or wherein R is

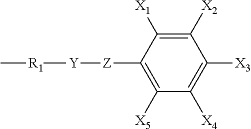

R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl.

In another embodiment, the current invention is drawn to a method of inhibiting α-N-acetylgalactosaminidases (GalNAcases) or β-hexosaminidases, comprising addition of a compound of the formula,

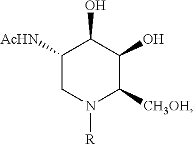

or a pharmaceutically acceptable salt thereof, wherein R is selected from the group consisting of hydrogen, substituted or unsubstituted alkyl groups, substituted or unsubstituted cycloalkyl groups, substituted or unsubstituted aryl groups, and substituted or unsubstituted oxaalkyl groups; or wherein R is

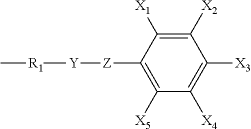

R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl, to a composition comprising α-N-acetylgalactosaminidases (GalNAcases) or β-hexosaminidases. In a further embodiment, R is hydrogen and the compound is DGJNAc.

In another embodiment, the current invention is drawn to a method of treating or preventing a disease associated with α-N-acetylgalactosaminidases (GalNAcases) or β-hexosaminidases activity comprising: administering to a subject in need thereof an effective amount of a compound of the formula,

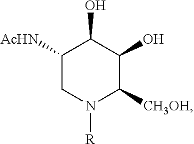

or a pharmaceutically acceptable salt thereof, wherein R is selected from the group consisting of hydrogen, substituted or unsubstituted alkyl groups, substituted or unsubstituted cycloalkyl groups, substituted or unsubstituted aryl groups, and substituted or unsubstituted oxaalkyl groups; or wherein R is

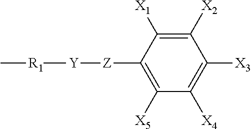

R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl,

In a further embodiment, the subject is a human being and the disease is Schindler disease. In addition, R may be hydrogen and the compound DGJNAc

### DETAILED DESCRIPTION

i. Iminosugars

Iminosugars, compounds in which the ring pyranose or furanose oxygen has been replaced by nitrogen, are the archetype for interaction with carbohydrate processing enzymes. (1). However, among the myriad of sugar mimics reported, there is not a single example of efficient inhibition of α-N-acetyl-galactosaminidases (GalNAcases).

ii. Overview

The current invention reports DGJNAc 1D and its derivatives as the first potent, specific and competitive inhibitors of GalNAcases. In addition, D-glucuronolactone 2D, a well-established chiron for the synthesis of many homochiral targets including amino acids (2) and iminosugars, (3) can be used as the starting material for an efficient synthesis of DGJNAc [2-acetamido-1,2-dideoxy-D-galacto-nojirimycin] 1D in an overall yield of 20%. The L-enantiomers of many iminosugars have surprising biological activities compared to their D-natural products. (4) The synthesis of L-DGJNAc 2L from the readily available (5) L-glucuronolactone 2L is also provided. The only previous synthesis of 1D starts from deoxynojirimycin (6) and a racemic mixture of 1D and 1L has also been prepared. (7) However, no investigations of the glycosidase inhibitory properties of 1D have previously been identified.

iii. Synthesis of DGJNAc from Glucuronolactone

The synthesis of DGJNAc1D requires introduction of nitrogen at C5 of glucuronolactone (Scheme 1), inversion of configuration of the hydroxyl group at C3 and formation of the piperidine ring by introduction of nitrogen between C6 and C2 (with inversion of configuration).

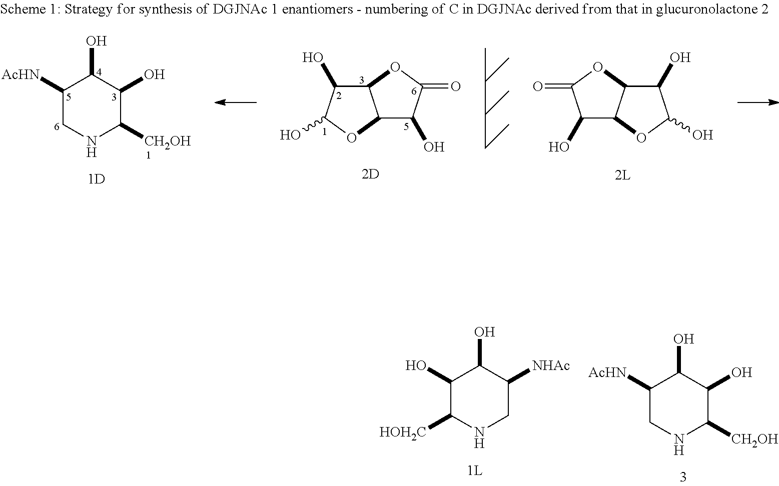

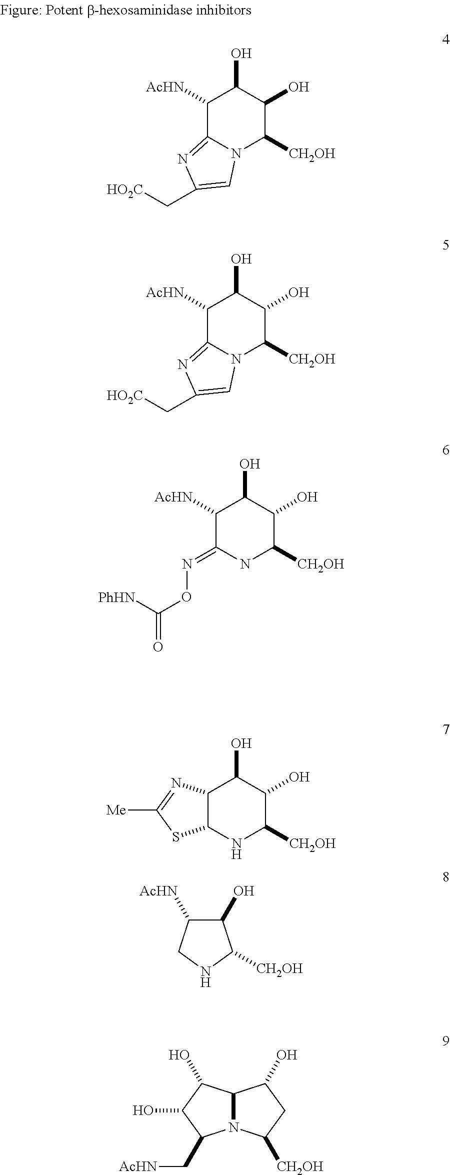

iv. Schindler Disease

Schindler disease, a congenital metabolic disorder, is a lysosomal storage disorder caused by a deficiency in the alpha-NAGA (alpha-N-acetylgalactosaminidase) enzyme. This lysosomal storage disorder is also known as Kanzaki disease and Alpha-N-acetylgalactosaminidase deficiency. Mutations to the NAGA gene on chromosome 22 lead to a build up of glycoproteins in the lysosomes and an accumulation of glycosphingolipids throughout the body. This accumulation of sugars causes the clinical features associated with this disease. Schindler disease is an autonomic recessive disorder.

There are three main types of the disease. In the Type I infantile form, babies develop normally until about a year old. Afterwards, the child begins to lose previously acquired skills associated with the coordination of physical and mental behaviors. Additional neurological and neuromuscular symptoms including diminished muscle tone, weakness, involuntary rapid eye movements, vision loss, and seizures may be present. Over time symptoms worsen and children experience a decreased ability to move certain muscles due to muscle rigidity and the ability to respond to external stimuli decreases. Other symptoms include neuroaxonal dystrophy from birth, discoloration of skin, Telangiectasia or widening of blood vessels.

In Type II adult form, symptoms are milder and may not appear until the mid 30 s. Angiokeratomas, an increased coarsening of facial features, and mild intellectual impairment are typical symptoms. Type III form is considered an intermediate disorder with varying symptoms among patients. Severe symptoms include seizures and mental retardation. Less severe symptoms include delayed speech, mild autistic like presentation, and/or behavioral problems.

v. β-Hexosaminidases

Other lysosomal enzymes are also well known for their role in numerous diseases. One example of these enzymes are β-hexosaminidases. Selective inhibition of β-hexosaminidases are useful for the study of osteoarthritis, (8) allergy, (9) Alzheimer's disease, (10) O-GlcNAcase inhibition, (11) cancer metastasis, (12) type II diabetes, (13) genetic diseases such as Tay-Sachs and Sandhoff diseases, (14) and of plant regulation. (15) The synthetic piperidine analogue of N-acetylglucosamine DGJNAc 3 (16) and its N-alkyl derivatives (17) are potent inhibitors of β-hexosaminidases. The natural product nagstatin 4, (18) with a galacto-configuration, does not inhibit GalNAcases even though it is a potent inhibitor of β-hexosaminidases. (19) The synthetic analogue with a gluco-configuration 5 (20) together with PUG derivatives 6 (21) and GlcNAc-thiazolines 7 (22) are very potent inhibitors of β-hexosaminidases. A rare example of a pyrrolidine potent hexosaminidase inhibitor is LABNAc 8; (23) the first pyrrolizidine β-hexosaminidase inhibitor, pochonicine 9 [or its enantiomer], has been isolated from a fungal strain Pochonia suchlasporia var. suchlasporia TAMA 87. (24) Some seven membered-ring imino sugars also display potent inhibition. (25)

Unless otherwise specified “a” or “an” means one or more.

vi. DGJNAc and its DGJNAc Derivatives

The present inventors discovered that certain iminosugars, such as DGJNAc and DGJNAc derivatives, may be effective in the inhibition of GalNAcases or β-hexosaminidases. In particular, the DGJNAc and DGJNAc derivatives may be useful for treating or preventing a disease or condition caused by or associated with GalNAcases) or β-hexosaminidases.

In many embodiments, the iminosugar is DGJNAc or a DGJNAC derivative. DGJNAc (2-acetamido-1,2-dideoxy-D-galacto-nojirimycin) is a compound of the formula

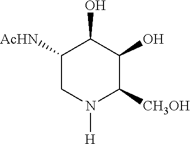

A “DGJNAc derivative” is a derivative of DGJNAc wherein the ring nitrogen is not substituted with a hydrogen atom.

In general, DGJNAc and DGJNAc derivatives can be represented by the formula

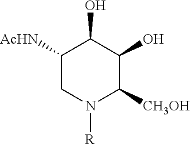

wherein R may be selected from hydrogen (for DGJNAc), substituted or unsubstituted alkyl groups, substituted or unsubstituted cycloalkyl groups, substituted or unsubstituted aryl groups, or substituted or unsubstituted oxaalkyl groups.

In some embodiments, R may be substituted or unsubstituted alkyl groups and/or substituted or unsubstituted oxaalkyl groups comprise from 1 to 16 carbon atoms, from 4 to 12 carbon atoms or from 8 to 10 carbon atoms. The term “oxaalkyl” refers to an alkyl derivative, which may contain from 1 to 5 or from 1 to 3 or from 1 to 2 oxygen atoms. The term “oxaalkyl” includes hydroxyterminated and methoxyterminated alkyl derivatives.

In some embodiments, R may be selected from, but is not limited to —(CH₂)₆OCH₃, —(CH₂)₆OCH₂CH₃, —(CH₂)₆O(CH₂)₂CH₃, —(CH₂)₆O(CH₂)₃CH₃, —(CH₂)₂O(CH₂)₅CH₃, —(CH₂)₂O(CH₂)₆CH₃; —(CH₂)₂O(CH₂)₇CH₃; —(CH₂)₉—OH; —(CH₂)₉OCH₃.

In some embodiments, R may be an branched or unbranched, substituted or unsubstituted alkyl group, which may contain up 20 carbon atoms. In some embodiments, the alkyl group may C2-C12 or C3-C7 alkyl group.

In certain embodiments, the alkyl group may be a long chain alkyl group, which may be C6-C20 alkyl group; C8-C16 alkyl group; or C8-C10 alkyl group. In some embodiments, R may be a long chain oxaalkyl group, i.e., a long chain alkyl group, which may contain from 1 to 5 or from 1 to 3 or from 1 to 2 oxygen atoms.

In some embodiments, R may have the following formula

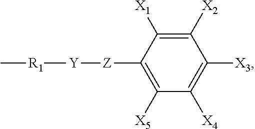

where R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl.

In some embodiments, Z is NH and R₁—Y is a substituted or unsubstituted alkyl group, such as C2-C20 alkyl group or C4-C12 alkyl group or C4-C10 alkyl group.

In some embodiments, X₁ is NO₂ and X₃ is N₃. In some embodiments, each of X₂, X₄ and X₅ is hydrogen.

vii. Salts, Prodrugs, Pharmaceutical Compositions

In some embodiments, the iminosugar may be in a form of a salt derived from an inorganic or organic acid. Pharmaceutically acceptable salts and methods for preparing salt forms are disclosed, for example, in Berge et al. (J. Pharm. Sci. 66:1-18, 1977). Examples of appropriate salts include but are not limited to the following salts: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

In some embodiments, the iminosugar may also used in a form of a prodrug.

In some embodiments, the iminosugar may be used as a part of a composition, which further comprises a pharmaceutically acceptable carrier and/or a component useful for delivering the composition to an animal. Numerous pharmaceutically acceptable carriers useful for delivering the compositions to a human and components useful for delivering the composition to other animals such as cattle are known in the art. Addition of such carriers and components to the composition of the invention is well within the level of ordinary skill in the art.

In some embodiments, the pharmaceutical composition may consist essentially of DGJNAc or the DGJNAc derivative, indicating that the DGJNAc or the DGJNAc derivative is the only active ingredient in the composition.

Yet in some other embodiments, DGJNAc or the DGJNAc derivative may be administered with one or more additional compounds.

In some embodiments, the iminosugar may be used in a liposome composition, such as those disclosed in US publication 2008/0138351; U.S. application Ser. No. 12/410,750 filed Mar. 25, 2009 and U.S. provisional application No. 61/202,699 filed Mar. 27, 2009.

viii. Administration and Inhibition of GalNAcases or β-hexosaminidases

The iminosugar, such as a DGJNAc or the DGJNAc derivative, may be administered to a cell or an animal affected by disorders associated with GalNAcases or β-hexosaminidases activity. The iminosugar may inhibit GalNAcases or β-hexosaminidases and help reduce, abate, or diminish the disease in the animal.

In addition, DGJNAc or the DGJNAc derivative, may be used to study the inhibition of GalNAcases or β-hexosaminidases with in vitro or in vivo studies.

Animals suffering from the disease include primates including monkeys and humans.

The amount of iminosugar administered to an animal or to an animal cell to the methods of the invention can be an amount effective to inhibit the GalNAcases or β-hexosaminidases. The term “inhibit” as used herein may refer to the detectable reduction and/or elimination of a biological activity exhibited in the absence of the iminosugar. The term “effective amount” may refer to that amount of the iminosugar necessary to achieve the indicated effect. The term “treatment” as used herein may refer to reducing or alleviating symptoms in a subject, preventing symptoms from worsening or progressing, inhibition or elimination of the causative agent, or prevention of the disorder related to the GalNAcases or β-hexosaminidases activity in a subject.

The amount of the iminosugar which may be administered to the cell or animal is preferably an amount that does not induce toxic effects which outweigh the advantages which accompany its administration.

Actual dosage levels of active ingredients in the pharmaceutical compositions may vary so as to administer an amount of the active compound(s) that is effective to achieve the desired therapeutic response for a particular patient.

The selected dose level may depend on the activity of the iminosugar, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient can depend on a variety of factors, including the body weight, general health, diet, time and route of administration and combination with other therapeutic agents and the severity of the condition or disease being treated. The adult human daily dosage may range from between about one microgram to about one gram, or from between about 10 mg and 100 mg, of the iminosugar per 10 kilogram body weight. Of course, the amount of the iminosugar which should be administered to a cell or animal may depend upon numerous factors well understood by one of skill in the art, such as the molecular weight of the iminosugar and the route of administration.

Pharmaceutical compositions that are useful in the methods of the invention may be administered systemically in oral solid formulations, ophthalmic, suppository, aerosol, topical or other similar formulations. For example, it may be in the physical form of a powder, tablet, capsule, lozenge, gel, solution, suspension, syrup, or the like. In addition to the active agent, such pharmaceutical compositions may contain pharmaceutically-acceptable carriers and other ingredients known to enhance and facilitate drug administration. Other possible formulations, such as nanoparticles, liposomes resealed erythrocytes, and immunologically based systems may also be used to administer the agent. Such pharmaceutical compositions may be administered by a number of routes. The term “parenteral” used herein includes subcutaneous, intravenous, intraarterial, intrathecal, and injection and infusion techniques, without limitation. By way of example, the pharmaceutical compositions may be administered orally, topically, parenterally, systemically, or by a pulmonary route.

These compositions may be administered in a single dose or in multiple doses which are administered at different times.

The present invention can be illustrated in more details by the following example, however, it should be understood that the present invention is not limited thereto.

Embodiments described herein are further illustrated by, though in no way limited to, the following working examples.

### EXAMPLE

### Example 1

### Synthesis of DJGNAc 1D from D-glucuronolactone 2D

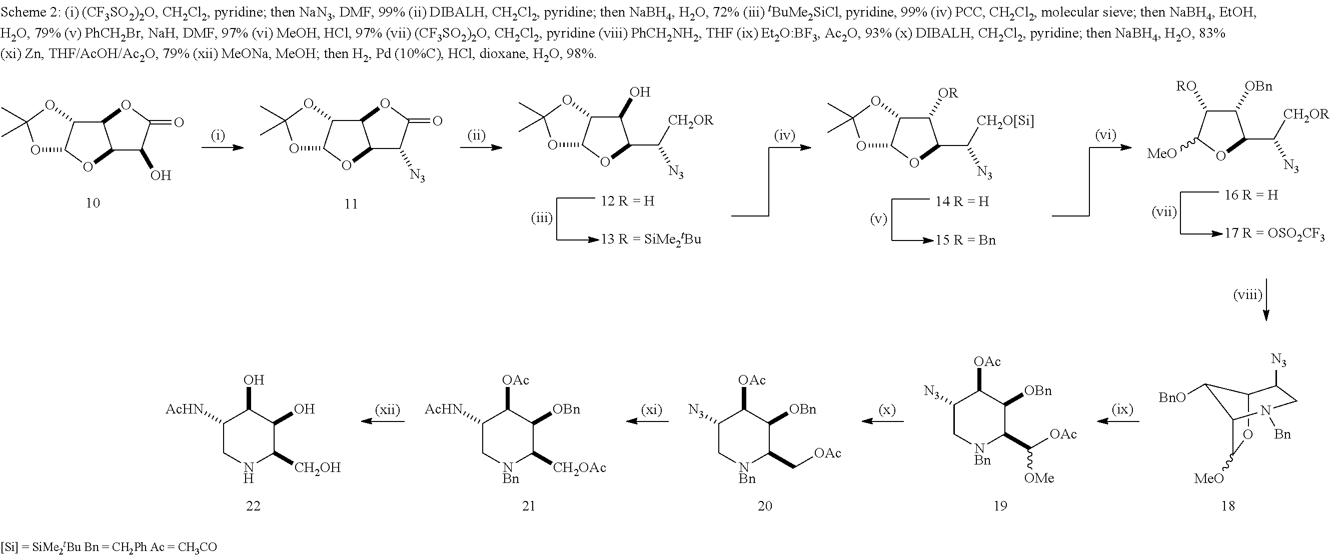

For the synthesis of DJGNAc 1D from D-glucuronolactone 2D, the acetonide 10 was esterified with trifluoromethanesulfonic(triflic)anhydride in dichloromethane in the presence of pyridine and the resulting crude triflate was treated with sodium azide in DMF to give the ido-azide 11 {mp. 112-114° C.; [α]_{D}²⁵+261.4 (c 1.0, CHCl₃) [lit. (29) mp. 114-116+ C., [α]_{D}²⁰ +243 (c 1.1, CHCl₃)]} in 97% yield. Direct conversion of the azidolactone 11 by a number of hydrides to the diol 12 gave only low yields; such α-azidolactones are extremely sensitive to base and commonly a two step reduction is necessary with initial reduction to the lactol. Accordingly DIBALH reduction of the azidolactone 11 in dichloromethane gave the corresponding lactol which was further reduced by sodium borohydride in methanol to afford the diol 12 mp. 120-122° C., [α]_{D}²⁵−69.6 (c 0.94, CHCl₃) in 72% yield. Selective protection of the primary alcohol in 12 by reaction with tert-butyldimethylsilyl (TBDMS) chloride gave the corresponding TBDMS ether 13, oil, [α]_{D}²⁵−12.7 (c 1.1, CHCl₃) in 99% yield; the overall yield of 13 from glucuronolactone 2D was 72% on a multigram scale and without any need for chromatographic purification until the final stage.

The synthesis of DGJNAc 1D required inversion and subsequent protection of the remaining unprotected C3 OH in the silyl ether 13. Oxidation of 13 with pyridinium chlorochromate in dichloromethane in the presence of molecular sieve afforded the corresponding ketone which on reduction from the least hindered face of the carbonyl gave the inverted alcohol 14, oil, [α]_{D}²⁵+74.9 (c 0.94, CHCl₃), in 79% yield. Treatment of 14 with benzyl bromide and sodium hydride in DMF formed the fully protected benzyl ether 15, oil, [α]_{D}²⁵+101.5 (c 0.56, CHCl₃) in 97% yield. Both the acetonide and silyl protecting groups in 15 were removed by treatment with HCl in methanol to give a 5:1 mixture of anomers of the methyl furanoside 16 (97%); reaction of 16 with triflic anhydride in dichloromethane in the presence of pyridine gave the ditriflate 17 which, with benzylamine in THF, gave the bicyclic pyrrolidine 18, oil, [α]_{D}²⁵+22.8 (c 1.11, CHCl₃), as a single anomer in an overall yield of 61%. Formation of a piperidine ring by cyclization of a ditriflate was thus efficient; examples of successful cyclizations of a ditriflate, such as the formation of a pyrrolidine, (30) are very rare.

Acetolysis of the furanoside 18 with boron trifluoride etherate in acetic anhydride gave a 4:1 mixture of the epimers 19 in 93% yield. The OMe group in 19 was reductively removed by sequential treatment with DIBALH in dichloromethane followed by sodium borohydride in methanol; acetylation of the resulting diol allowed easy isolation of the diacetate 20, oil, [α]_{D}²⁵+79.8 (c 0.43, CHCl₃), in 83% overall yield from 19. Rapid reduction of the azide in XK by zinc dust in the presence of copper(II) sulfate in acetic acid-acetic anhydride-THF (31) with subsequent acylation of the corresponding amine gave the crystalline triacetate 21, mp. 112-114° C., [α]_{D}²⁵+26.2 (c 1.1, Me₂CO) in 79% yield. Removal of the acetate protecting groups by treatment of 21 with sodium methoxide in methanol followed by hydrogenolysis of the benzyl groups by palladium (10% on carbon) in dioxane: aqueous hydrochloric acid gave DGJNAc 1D,mp. 150-154° C., [α]_{D}²⁵+41.9 (c 0.67, H₂O)) [lit.⁶ oil, [α]_{D}²⁰+37 (c 1, MeOH)], in 98% yield. Unlike many iminosugars, the free base DGJNAc is readily crystallized; the overall yield of DGJNAc 1D from D-glucuronolactone 2D was 20%.

Selected data for DGJNAc 1D: HRMS (ESI+ve): C₈H₁₆N₂NaO₄ found 227.1001; (M+Na⁺) requires 227.1002; +41.9 (c 0.67, H₂O); m.p. 150-154° C.; νₘₐₓ (thin film, Ge): 3287 (br, s, OH/NH), 1637 (s, amide I), 1561 (s, amide II); δ_{H} (D₂O, 400 MHz): 2.00 (3H, s, Me), 2.37 (1H, dd, H1a J_{gem} 12.9, J_{1a,2} 11.6), 2.76 (1H, dt, H5 J_{5,4} 1.3, , J_{5,6a}=J_{5,6b} 6.6), 3.08 (1H, dd, H1b J_{gem} 12.9, J_{1b,2} 5.1), 3.58 (1H, dd, H3 J_{3,2} 10.6, J_{3,4} 3.0), 3.61 (1H, dd, H6a J_{gem} 11.1, J_{6a,5} 6.3), 3.65 (1H, dd, H6b J_{gem} 11.1, J_{6b,5} 6.6), 3.96 (1H, dt, H2 J_{2,1a} 11.1, J_{2,1b} 5.1, J_{2,3} 11.1), 4.01 (1H, dd, H4 J_{4,3} 3.0, J_{4,5} 1.4); δ_{C} (D₂O, 100 MHz): 22.7 (Me), 47.7 (Cl), 49.1 (C2), 59.4 (C5), 61.9 (C6), 68.9 (C4), 73.2 (C3), 175.2 (COMe); LRMS (ESI+ve): 205 (77%, M+H⁺), 431 (100%, 2M+Na⁺).

### Example 2

### Synthesis of L-DJGNAc 1L from L-glucuronolactone 2L

The enantiomer L-DGJNAc 1L, mp. 152-156° C., [α]_{D}²⁵ 46.6 (c 0.73, H₂O), was prepared by an identical procedure from L-glucuronolactone 2L.

### Example 3

### Inhibition of GalNAcases and β-hexosaminidases by DJGNAc 1D and L-DGJNAc 1L

DGJNAc 1D was a highly potent competitive inhibitor of GalNAcases (Kᵢ 0.081 μM from chicken liver, Kᵢ 0.136 μM from Charonia lampas); DGJNAc 1D was a good but much less potent competitive inhibitor of β-hexosaminidases (IC₅₀ 1.8 μM from Jack bean, IC₅₀ 1.8 μM from Jack bean, IC₅₀ 4.2 μM from bovine kidney, IC₅₀ 8.3 μM from human placenta, IC₅₀ 2.2 μM from HL-60).

The enantiomer L-DGJNAc 1L, showed no inhibition of α-N-acetylgalactosaminidases but was a very weak but non-competitive inhibitor of β-hexosaminidases [Kᵢ 1100 μM—compared with Kᵢ 2.2 μM for DGJNAc 1D—from human placenta]. This result was in accord with Asano's hypothesis (32) that L-enantiomers show non-competitive inhibition whereas D-imino sugars usually are competitive inhibitors.

DGJNAc 1D showed modest inhibition of coffee bean α-galactosidase (IC₅₀ 64 μM) whereas L-DGJNAc 1L, showed no inhibition of this enzyme.

Both enantiomers of DGJNAc 1 were screened as inhibitors of a number of other glycosidases and neither enantiomer showed any significant inhibition [less than 50% inhibition at 1000 mM) against α-glucosidases (rice, yeast), β-glucosidases (almond, bovine liver), β-galactosidase (bovine liver), α-mannosidase (Jack bean), β-glucuronidases (E. coli, bovine liver), α-L-rhamnosidase (P. decumbens), or α-L-fucosidase (bovine epididymis).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

### REFERENCES

1. (a) Asano, N. Cell. Mol. Life Sci. 2009, 66, 1479-1492. (b) Compain, P.; Martin, O. R. Iminosugars: from synthesis to therapeutic application, ISBN-0-470-03391-3, John Wiley & Son, 2007. (c) Asano, N.; Nash, R. J.; Molyneux, R. J.; Fleet, G. W. J. Tetrahedron: Asymmetry 2000, 11, 1645-1680. (d) Watson, A. A.; Fleet, G. W. J.; Asano, N.; Molyneux, R. J.; Nash, R. J. Phytochemistry 2001, 56, 265-295.
2. (a) Bashyal, B. P.; Chow, H.-F.; Fellows, L. E.; Fleet, G. W. J. Tetrahedron, 1987, 43, 423-430. (b) Bashyal, B. P.; Chow, H.-F.; Fleet, G. W. J. Tetrahedron Lett., 1986, 27, 3205-3208.
3. (a) Anzeveno, P. B.; Creemer, L. J. Tetrahedron Lett. 1990, 31, 2085-2088. (b) Klemer, A.; Hofmeister, U.; Lemmes, R. Carbohydr. Res. 1979, 68, 391-5. (c) Paulsen, H.; Guenther, C. Chem. Ber. 1977, 110, 2150-2157. (d) Best, D.; Chen Wang, C.; Weymouth-Wilson, A. C.; Clarkson, R. A.; Wilson, F. X.; Nash, R. J.; Miyauchi, S.; Kato, A.; Fleet, G. W. J. Tetrahedron: Asymmetry 2010, 21, SUBMITTED FOR PUBLICATION.
4. (a) D'Alonzo, D.; Guaragna; A.; Palumbo, G. Curr. Med. Chem. 2009, 16, 473-505. (b) Clinch, K.; Evans, G. B.; Fleet, G. W. J.; Furneaux, R. H.; Johnson, S. W.; Lenz, D.; Mee, S.; Rands, P. R.; Schramm, V. L.; Ringia, E. A. T.; Tyler, P. C. Org. Biomol. Chem. 2006, 4, 1131-1139. (c) Smith, S. S. Toxicol. Sci. 2009, 110, 4-30. (d) Mercer, T. B.; Jenkinson, S. F.; Nash, R. J.; Miyauchi, S.; Kato, A.; Fleet, G. W. J. Tetrahedron: Asymmetry 2009, 20, 2368-2373.
5. Weymouth-Wilson, A. C.; Clarkson, R.; Best, D.; Pino-Gonzalez, M.-S.; Wilson, F. X.; Fleet, G. W. J. Tetrahedron Lett. 2009, 50, 6307-6310.
6. Schueller, A. M.; Heiker, F. R. Carbohydr. Res. 1990, 203, 308-313.
7. Kang, S. H; Ryu, D. H. Tetrahedron Lett. 1997, 38, 607-610.
8. Liu, J.; Numa, M. M. D.; Huang, S.-J.; Sears. P.; Shikhman, A. R.; Wong, C.-H. J. Org. Chem. 2004, 69, 6273-6283.
9. Reese, T. A.; Liang, H.-E.; Tager, A. M.; Luster, A. D.; van Roojen, N.; Voehringer, D.; Locksley, R. M. Nature 2007, 447, 92-96.
10. Liu, F.; Iqbal, K.; Grundje-Iqbal, I.; Hart, G. W.; Gong, C.-X. Proc. Natl. Acad. Sci. 204, 101, 10804-10809.
11. (a) Wells, L.; Voseller, K.; Hart, G. W. Science 2001, 291, 2376-2378. (b) Hanover, J. A. FASEB J. 2001, 15, 1865-1876.
12. (a) Woynarowska, B.; Wikiel, H.; Sharma, M.; Fleet, G. W. J.; Bernacki, R. J. Proc. Amer. Assoc. Cancer Res. 1989, 30, 91. (b) Woynarowska, B.; Wikiel, H.; Sharma, M.; Carpenter, N.; Fleet, G. W. J.; Bernacki, R. J. Anticancer Res. 1992, 12, 161-166.
13. Voseller, K.; Wells, L.; Lane, M. D.; Hart, G. W. Proc. Natl. Acad. Sci. 2002, 99, 5315-5318.
14. (a) Kolter, T.; Sandhoff, K. Biochem. Biophys. Acta 2006, 1758, 2057-2079. (b)) Kolter, T.; Sandhoff, K. Angew. Chem. Int. Ed. 1999, 38, 1532-1568.
15. Horsch, M.; Hoesch, L.; Fleet, G. W. J.; Rast, D. M. J. Enzyme Inhibition 1993, 7, 47-53.
16. (a) Fleet, G. W. J.; Fellows, L. E.; Smith, P. W. Tetrahedron 1987, 43, 979-990. (b) Fleet, G. W. J.; Smith, P. W.; Nash, R. J.; Fellows, L. E.; Parekh, R. B.; Rademacher, T. W. Chem. Lett. 1986, 1051-1054. (c) Boshagen, H.; Heiker, F.-R.; Schueller, A. M. Carbohydr. Res. 1987, 164, 141-148.
17. Steiner, A. J.; Schitter, G.; Stutz, A. E.; Wrodnigg, T. M.; Tarling, C. A.; Withers, S. G.; Mahuran, D. J.; Tropak, M. B., Tetrahedron Aymmetry 2009, 20, 832-835.
18. Aoyama, T.; Naganawa, H.; Suda, H.; Uotani, K.; Aoyagi, T.; Takeuchi, T. J. Antibiot. 1992, 45, 1557-1558.
19. Tatsuta, K.; Miura, S.; Gunji, H. Bull. Chem. Soc. Jpn. 1997, 70, 427-436. (c) Takahashi, S.; Terayama, H.; Kuzuhara, H. Tetrahedron 1996, 52, 13315-13326. (d) Tatsuta, K.; Miura, S. Tetrahedron Lett. 1995, 36, 6721-6724. (e) Tatsuta, K.; Miura, S.; Ohta, S.; Gunji, H. J. Antibiot. 1995, 48, 286-288.
20. Dorfmueller, H. C.; Borodkin, V. S.; Schimpl, M.; van Aalten, D. M. F. Biochem. J. 2009, 420, 221-227.
21. Shanmugasundaram, B.; Debowski, A. W.; Dennis, R. J.; Davies, G. J.; Vocadlo, D. J.; Vasella, A. Chem. Commun. 2006, 4372-4374.
22. (a) Knapp, S.; Fash, D.; Abdo, M.; Emge, T. J.; Rablen, P. R. Bioorg. Med. Chem. 2009, 17, 1831-1836. (b) Knapp, S.; Vocadlo, D.; Gao, Z. N.; Kirk, B.; Lou, J. P.; Withers, S. G. J. Amer. Chem. Soc. 1996, 118, 6804-6805.
23. (a) Rountree, J. S. S.; Butters, T. D.; Wormald, M. R.; Dwek, R. A.; Asano, N.; Ikeda, K.; Evinson, E. L.; Nash, R. J.; Fleet, G. W. J. Tetrahedron Lett. 2007, 48, 4287-4291. (b) Rountree, J. S. S.; Butters, T. D.; Wormald, M. R.; Boomkamp, S. D.; Dwek, R. A.; Asano, N.; Ikeda, K.; Evinson, E. L.; Nash, R. J.; Fleet, G. W. J. ChemMedChem 2009, 4, 378-392.
24. Usuki, H.; Toyo-oka, M.; Kanzaki, H.; Okuda, T.; Nitoda, T. Bioorg. Med. Chem. 2009, 17, 7248-7253.
25. Li, H. Q.; Marcelo, F.; Bello, C.; Vogel, P.; Butters, T. D.; Rauter, A. P.; Zhang, Y. M.; Sollogoub, M.; Bleriot, Y. Bioorg. Med. Chem. 2009, 17, 5598-5604.
26. (a) Clark, N. E.; Garman, S. C. J. Mol. Biol. 2009, 393, 435-447. (b) Kanekura, T.; Sakuraba, H.; Matsuzawa, F.; Aikawa, S.; Doi, H.; Hirabayashi, Y.; Yoshii, N.; Fukushige, T.; Kanzaki, T. J. Dermatol. Sci. 2005, 37, 15-20. (c) Chabas, A.; Duque, J.; Gort, L. J. Inherited Metabol. Disease 2007, 30, 108-108. (d) Staretz-Chacham, O.; Lang, T. C.; LaMarca, M. E.; Krasnewich, D.; Sidransky, E. Pediatrics 2009, 123,1191-1207. (e) Asfaw, B.; Ledinova, J.; Dobrovolny, R.; Bakker, H. D.; Desnick, R. J.; van Diggelen, O. P.; de Jong, J. G. N.; Kanzaki, T.; Chabas, A.; Maire, I.; Conzelmann, E.; Schindler, D. J. Lipid Res. 2002, 43, 1096-1104.
27. (a) Greco, M.; De Mitri, M.; Chiriaco, F.; Leo, G.; Brienza, E.; Maffia, M. Cancer Lett. 2009, 283, 222-229. (b) Yin, D. S.; Ge, Z. Q.; Yang, W. Y.; Liu, C. X.; Yuan, Y. J. Cancer Lett. 2006, 243, 71-79. (c) Mohamad, S. B.; Nagasawa, H.; Uto, Y.; Hori, H. Comparative Biochem. Physiol. A: Molec. Intergrative Physiol. 2003, 134, 481-481. (d) Bin Mohamad, S.; Nagasawa, H.; Uto, Y.; Hori, H. Anticancer Res. 2002, 22, 4297-4300.
28. (a) Willis, L. M.; Zhang, R.; Reid, A.; Withers, S. G.; Wakarchuk, W. W. Biochem. 2009, 48, 10334-10341. (b) Suzuki, R.; Katayama, T.; Kitaoka, M.; Kumagai, H.; Wakagi, T.; Shoun, H.; Ashida, H.; Yamamoto, K.; Fushinobu, S. J. Biochem. 2009, 146, 389-398. (c) Marion, C.; Limoli, D. H.; Bobulsky, G. S.; Abraham, J. L.; Burnaugh, A. M.; King, S. J. Infect. Immun. 2009, 77, 1389-1396. (d) Goda, H. M.; Ushigusa, K.; Ito, H.; Okino, N.; Narimatsu, H.; Ito, M. Biochem. Biophys. Res. Commun. 2008, 375, 541-546. (e) Koutsioulis, D.; Landry, D.; Guthrie, E. P. Glycobiology 2008, 18, 799-805.(f) Ashida, H.; Maki, R.; Ozawa, H.; Tani, Y.; Kiyohara, M.; Fujita, M.; Imamura, A.; Ishida, H.; Kiso, M.; Yamamoto, K. Glycobiology 2008, 18, 727-734.
29. Bashyal, B. P.; Chow, H.-F.; Fleet, G. W. J. Tetrahedron, 1987, 43, 415-422.
30. (a) Shing, T. K. M. J. Chem. Soc., Chem. Commun. 1987, 262-263. (b) Shing, T. K. M. Tetrahedron 1988, 7261-7264.
31. Campo, V. L.; Carvalho, I.; Allman, S.; Davis; B. G.; Field, R. A. Org. Biomol. Chem. 2007, 5, 2645-2657. (b) Winans, K. A.; King, D. S.; Rao, V. R.; Bertozzi, C. R. Biochemistry 1999, 38, 11700-11710.
32. (a) Kato, A.; Kato, N.; Kano, E.; Adachi, I.; Ikeda, K.; Yu, L.; Okamoto, T.; Banba, Y.; Ouchi, H.; Takahata, H.; Asano, N. J. Med. Chem. 2005, 48, 2036-2044. (b) Asano, N.; Ikeda, K.; Yu, L.; Kato, A.; Takebayashi, K.; Adachi, I.; Kato, I.; Ouchi, H.; Takahata, H.; Fleet, G. W. J. Tetrahedron: Asymmetry 2005, 16, 223-229.

What is claimed is:

1. A method for synthesizing DGJNAc or a DGJNAc derivative from D-glucuronolactone, comprising
introducing nitrogen at C5 of glucuronolactone,
inversion of the configuration of the hydroxyl group at C3, and
formation of the piperidine ring by introduction of nitrogen between C6 and C2.

2. A compound of the following formula, 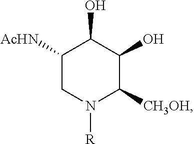
or a pharmaceutically acceptable salt thereof, wherein R is selected from the group consisting of substituted or unsubstituted alkyl groups, substituted or unsubstituted cycloalkyl groups, substituted or unsubstituted aryl groups, and substituted or unsubstituted oxaalkyl groups;
or wherein R is 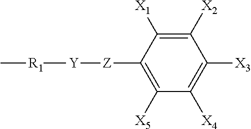
R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl.

3. A method of inhibiting α-N-acetylgalactosaminidases (GalNAcases) or β-hexosaminidases, comprising addition of a compound of the formula, 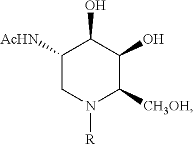
or a pharmaceutically acceptable salt thereof, wherein R is selected from the group consisting of hydrogen, substituted or unsubstituted alkyl groups, substituted or unsubstituted cycloalkyl groups, substituted or unsubstituted aryl groups, and substituted or unsubstituted oxaalkyl groups;
or wherein R is 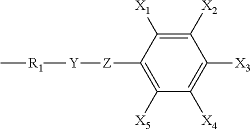
R₁ is a substituted or unsubstituted alkyl group;
X₁₋₅ are independently selected from H, NO₂, N₃, or NH₂;
Y is absent or is a substituted or unsubstituted C₁-alkyl group, other than carbonyl; and
Z is selected from a bond or NH; provided that when Z is a bond, Y is absent, and provided that when Z is NH, Y is a substituted or unsubstituted C₁-alkyl group, other than carbonyl,
to a composition comprising α-N-acetylgalactosaminidases (GalNAcases) or β-hexosaminidases.

4. The method of claim 3, where R is hydrogen.

5. A method of treating or preventing a disease associated with α-N-acetylgalactosaminidases (GalNAcases) or β-hexosaminidases activity comprising:
administering to a subject in need thereof an effective amount of a compound of the formula, 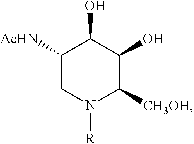
or a pharmaceutically acceptable salt thereof, wherein R is selected from the group consisting of hydrogen, substituted or unsubstituted alkyl groups, substituted or unsubstituted cycloalkyl groups, substituted or unsubstituted aryl groups, and substituted or unsubstituted oxaalkyl groups;
or wherein R is 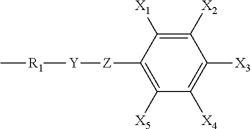
R₁ is a substituted or unsubstituted alkyl group;
X₁₋₅ are independently selected from H, NO₂, N₃, or NH₂;
Y is absent or is a substituted or unsubstituted Cᵢ-alkyl group, other than carbonyl; and
Z is selected from a bond or NH; provided that when Z is a bond, Y is absent, and provided that when Z is NH, Y is a substituted or unsubstituted C₁-alkyl group, other than carbonyl,

6. The method of claim 5, wherein the subject is a human being.

7. The method of claim 5, wherein said subject has Schindler Disease.

8. The method of claim 5, wherein R is hydrogen.
